(19) United States
(12) Patent Application Publication
(10) Pub. No.: US 2002/0038041 A1
(43) Pub. Date: Mar. 28, 2002

(54) Glycidyl carbonates

(75) Inventors: John H. Clements, Round Rock, TX (US); Howard P. Klein, Austin, TX (US); Edward T. Marquis, Austin, TX (US)

Correspondence Address: Russell R. Stolle, Huntsman Corporation, P.O. Box 15730, Austin, TX, 78761, US

(73) Assignee: Huntsman Petrochemical Corporation, Austin, TX

(21) Appl. No.: 09/946,318

(22) Filed: Sep. 5, 2001

Related U.S. Application Data
(63) Continuation-in-part of application No. 09/579,607, filed on May 26, 2000.

(51) Int. Cl. C 07D 3 3/40; C07D 301/14

(52) U.S. Cl. 549/525

(57) ABSTRACT

A method of preparing novel alkyl glycidyl carbonate compositions comprising reacting a metal alkoxide with a cyclic organic carbonate in the presence of a solvent under conditions suitable for producing an alkyl glycidyl carbonate. Novel alkyl glycidyl carbonate compositions are also disclosed.

### CROSS-REFERENCES TO RELATED APPLICATIONS

This Application is a Continuation-In-Part of and claims priority to, U.S. patent application Ser. No. 09/579,607 filed May 26, 2000, which is currently pending.

### TECHNICAL FIELD

This invention relates to organic carbonates. More particularly it relates to the preparation of alkyl glycidyl carbonate compositions.

### BACKGROUND OF THE INVENTION

The field of organic chemistry as it relates to adhesives, coatings, elastomers, and composites is broad indeed, encompassing many general classes of compounds useful as raw materials and intermediates in the manufacture of such finished products. It is well-known in the art that the organic carbonates: ethylene carbonate, propylene carbonate, and butylene carbonate, inter alia, are one general class of materials that are useful in such regard. For example, from ethylene carbonate one can derive β-hydroxyethyl thiol, or β-hydroxyalkyl thioethers, which are useful as specialty solvents. From ethylene carbonate one can also derive carboxylic acid esters, β-hydroxyethyl esters, for example, which are useful in modifying super-absorbing polymers and in modifying the acidity level of polyesters. From ethylene carbonate one can also derive β-hydroxyethyl alkylcarbonates, for example, which are useful in functional fluid formulations. From ethylene carbonate one can also derive dialkyl carbonates, such as diethyl carbonate, for example, which is useful in transesterification reactions. From ethylene carbonate one can also derive linear polycarbonate diols, which are useful in preparation of polyurethane elastomers, and as compatabilizers for supercritical carbon dioxide in a variety of applications. From ethylene carbonate one can also derive polyethylene phthalates (polyesters), for example, which are useful in plastic container manufacture, and in manufacture of coatings. Thus, the uses of alkylene carbonates are quite broad and varied.

We have discovered that when a halomethyl-substituted cyclic alkylene carbonate is subjected to reaction under certain conditions with a metallic alkoxide salt, that a reaction occurs which results in the formation of an alkyl glycidyl carbonate having the structure:

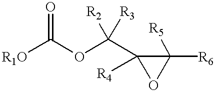

in which R₁ may be independently selected from the group consisting of: hydrogen or any alkyl group having any number of carbon atoms in the range of 1-12, (“inclusive”, i.e., including 1 and 12); and R₂, R₃, R₄, R₅, and R₆ may each be independently selected from the group consisting of: hydrogen or any alkyl group having 1, 2, 3, or 4 carbon atoms, depending upon the choice of starting material as later described herein. We believe that such materials are not described in any of the chemical literature, and represent a new class of compound from which it is possible to prepare many useful end products. For example, the neat material in which R₂, R₃, R₄, R₅, and R₆ are all hydrogen and R₁ is tertiary butyl is especially useful as a carbonate-functional polyoxyalkylene that can be converted to a graft copolymer via reaction with polyols. Such materials are chemically resistant resins useful in coatings and surfactants applications.

Now that we have identified this new class of compound, we suggest that those materials for which one or more of the R₂, R₃, R₄, R₅, and R₆ substituents comprise one or more of the various alkyl groups possible, should also be readily useful as resin when polymerized and reacted as above but which is more hydrophobic. For those cases where R₅ or R₆ is methyl, then the polymer backbone is reminiscent of (poly)propyleneoxide as opposed to (poly)ethyleneoxide as in the above example, as well as chemical intermediates for polymer applications where polymers are employed, such as coatings, adhesives, composites, and elastomers. The present invention provides these alkyl glycidyl carbonate compositions and methods for their preparation.

### SUMMARY OF THE INVENTION

The present invention is directed toward alkyl glycidyl carbonate compositions and their preparation. According to one embodiment of the present invention, alkyl glycidyl carbonate compositions may be prepared by reacting a metal alkoxide with a cyclic organic carbonate in the presence of a solvent under conditions suitable for producing an alkyl glycidyl carbonate. A cyclic organic carbonate capable of participating in such reaction has the general structure:

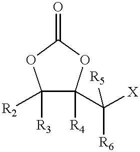

in which R₂, R₃, R₄, R₅, and R₆ are each independently a hydrogen or an alkyl group; and X is F, Cl, Br, I, or another substituent or group recognized by those skilled in the art as capable of acting as a leaving group in a nucleophilic substitution reaction. The material in which R₂, R₃, R₄, R₅, and R₆ are all hydrogen and in which X is a chlorine may be made by insertion of CO₂ into epichlorohydrin under conditions of high temperature and pressure, as later set forth in Example 1.

According to the invention, alkyl glycidyl carbonate compositions are prepared by reacting a metal alkoxide with a cyclic organic carbonate described above in the presence of a suitable solvent under conditions as herein described. Alkyl glycidyl carbonate compositions prepared according to the invention have the general structure:

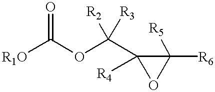

in which R₁ is any alkyl group having between 1 and 12 carbon atoms, including 1 and 12; and R₂, R₃, R₄, R₅, and R₆ are each independently selected from the group consisting of: hydrogen; or any alkyl group having 1, 2, 3, or 4 carbon atoms.

### DETAILED DESCRIPTION

The present invention provides alkyl glycidyl carbonates and methods for their preparation. According to one preferred form of the invention, alkyl glycidyl carbonates are prepared by reacting a metal alkoxide with a cyclic organic carbonate in the presence of a suitable solvent under conditions under which an appreciable yield of alkyl glycidyl carbonate is obtained. Suitable cyclic organic carbonate starting materials have the general structure:

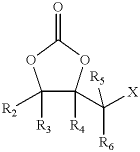

in which R₂, R₃, R₄, R₅, and R₆ are each independently a hydrogen or an alkyl group; and X is fluorine, chlorine, bromine, iodine, or any other group or atom generally recognized by organic chemists as being capable of functioning as a leaving group in a nucleophilic substitution reaction. Such other suitable leaving groups may comprise, but are not limited to, a phenoxy, alkylester, sulfate, sulfite, acetate, benzoate, or tosylate. Such cyclic organic carbonates are readily prepared from compounds such as epichlorohydrin and carbon dioxide, according to the reaction scheme:

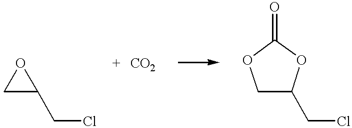

as disclosed in Example 1.

When using a preferred cyclic organic carbonate according to the invention, the net reaction according to the invention may be represented by the general equation:

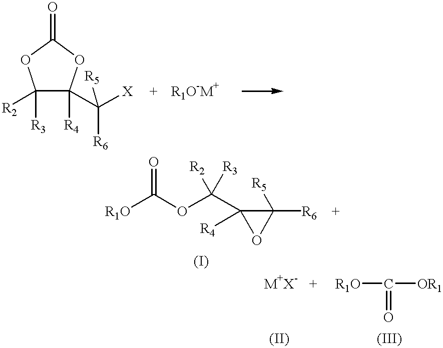

in which R₁ is an alkyl group; R₂, R₃, R₄, R₅, and R₆ are each independently a hydrogen or an alkyl group; M may be any metal atom from which alkoxides are known to have been prepared or which is capable of providing charge balance for an alkoxide or phenolate or other similar anion, and includes without limitation: lithium, potassium, sodium, rubidium, cesium, calcium, magnesium, strontium, aluminum, etc. The substituent X generally represents a halogen atom, including fluorine, chlorine, bromine, iodine, but may alternatively be any other group or atom generally recognized by organic chemists as being capable of functioning as a leaving group within the context of the nucleophillic substitution reaction. The products of the above reaction comprise: the alkyl glycidyl carbonate compositions of the present invention, (I); a salt, (II); and a minor amount of a dialkyl carbonate, (III). The reaction product may also comprise a minor amount of glycidol.

As in most organic reactions, in the production of alkyl glycidyl carbonates according to the present invention, there are several reaction variables which may be controlled to achieve the desired product. One such factor is the external pressure, which in the instant case may be atmospheric pressure, super-atmospheric pressure, or sub-atmospheric pressure. According to a preferred form of the invention, the reaction mixture is blanketed with an inert gas atmosphere, such as, but not limited to, a nitrogen or argon gas atmosphere, in order to minimize side-reactions and oxidations which might otherwise occur.

An important factor is the temperature at which the reaction is conducted. According to a preferred form of the invention, the reactants are maintained at a temperature below about 20 degrees centigrade during the course of the reaction. According to a more preferred form of the invention, the reactants are maintained at a temperature below about 0 degrees centigrade during the course of the reaction. The most preferred temperature range for carrying out a reaction according to the invention is any temperature between about −20 degrees Centigrade and −80 degrees Centigrade, which is preferably achieved by immersion of the reaction vessel in a dry ice/acetone bath, or other functionally equivalent means known to those skilled in the art. Generally the yield of alkyl glycidyl carbonate decreases with increasing reaction temperature, and unwanted by-products become a significant fraction of the reaction product. It is for this reason that the temperature is preferably maintained below about −20 degrees Centigrade during the course of the reaction.

While metal alkoxides, phenolates, and the like are fairly reactive towards the cyclic alkylene carbonate starting materials, it is nevertheless, desirable in general to employ a slight excess of the alkoxide, phenolate, or like material with respect to the cyclic alkylene carbonate starting material, since it is possible that a portion of the alkoxide, phenolate, or the like, being so reactive towards many things, including traces of moisture, etc., may be consumed somehow before it has the chance to react with the cyclic alkylene carbonate starting material. Thus it is preferred that the alkoxide, phenolate, or the like is present in an excessive amount with respect to the cyclic organic carbonate of between about 1% to about a 10% molar excess.

The cyclic organic carbonate reactant useful in accordance with the invention may be any cyclic organic carbonate. Thus, suitable cyclic organic carbonates include, without limitation: 4-iodomethyl-1,3-dioxolan-2-one; 4-bromomethyl-1,3-dioxolan-2-one; or 4-chloromethyl-1,3-dioxolan-2-one. A preferred product according to the invention is prepared using 4-chloromethyl-1,3-dioxolan-2-one as a reactant.

When a metal alkoxide is selected as a reactant to react with a cyclic alkylene carbonate according to the invention, any alkoxide which upon reaction with water yields an alcohol having between 1 and 12 carbon atoms, including methanol and dodecanols, whether straight-chain or branched, may be employed. However, it is preferred that the metal alkoxide comprises an alkoxide selected from the group consisting of: sodium t-butoxide; potassium t-butoxide; lithium t-butoxide; sodium ethoxide; sodium propoxide; sodium isopropoxide; potassium ethoxide; lithium ethoxide; sodium methoxide; potassium methoxide; or lithium methoxide. According to a preferred form of the invention, the metal alkoxide comprises sodium t-butoxide.

A reaction for preparing alkyl glycidyl carbonates according to the invention is preferably carried out in the presence of a suitable solvent. As is known generally to those skilled in the art of organic chemistry, it is common to employ as a solvent the same alcohol from which an alkoxide anion is derivable, when alkoxide anions are employed as reactants. Thus, in cases where an ethoxide ion is employed, the solvent of choice is often ethanol. In cases where an isopropoxide ion is employed, the solvent of choice is often isopropanol, and so on. Therefore, alcohols having between 1 and 12 carbon atoms, including methanol and dodecanols, whether straight-chain or branched, may be employed as a solvent in conducting a reaction according to the invention.

During the practice of the reaction set forth herein in preparing alkyl glycidyl carbonates, the solvent may further comprise ethers, in addition to alcohols, either alone, or in combination with one or more other ethers or an alcohol. Suitable ether solvents include, but are not limited to, tetrahydrofuran, methyl tert-butyl ether (MTBE), diethyl ether, dimethyl ether, or ethylene glycol dimethyl ether. When an ether is selected as a solvent, it preferably comprises tetrahydrofuran (THF).

The alkyl glycidyl carbonate products produced in accordance with the present invention may be recovered and isolated using any number of suitable purification methods known to those skilled in the art of organic chemistry. For example, when R₁ is t-butyl, the alkyl glycidyl carbonate product may be recovered and isolated by first removing a significant portion of the ether solvent by rotary evaporation, and then isolating the alkyl glycidyl carbonate using water/diethyl ether separation. For further purification, following this initial separation, the organic phase may then be washed several times with water, dried (MgSO₄), and filtered. In an alternate scheme, useful, inter alia, when R₁ is an ethyl, methyl, or t-butyl, the alkyl glycidyl carbonate product may be recovered and isolated by first adding water (about 1% by volume of the total solution), followed by addition of an alkaline earth silicate such as Magnesol® (available from the Dallas Group, Inc.) in an amount approximately equal to the weight of the expected amount of M⁺X⁻produced during the reaction. The resulting mixture is then stirred and filtered, and then the solvents are removed by evaporation using a Rotovap®. Regardless of the method used for the initial separation, further purification may be achieved by distillation at a temperature of about 50° C. to about 60° C., and at a pressure of about 0.1 mmHg to about 0.5 mmHg to yield a pure alkyl glycidyl carbonate product.

The present invention provides novel compositions of matter, which we term alkyl glycidyl carbonates, which have the general structure:

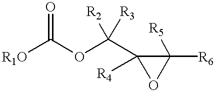

in which R₁ is any alkyl group having between 1 and 12 carbon atoms; and R₂, R₃, R₄, R₅, and R₆ are each independently selected from the group consisting of: hydrogen; or any alkyl group having between 1 and 4 carbon atoms. According to one preferred form of the invention, R₁ is a methyl, ethyl, or t-butyl. In another preferred form of the invention, R₁ is t-butyl, and R₂, R₃, R₄, R₅, and R₆ are each hydrogen.

The alkyl glycidyl carbonates of the present invention may be polymerized to one or more terminal ends of a polyoxyalkylene composition according to the generic equation:

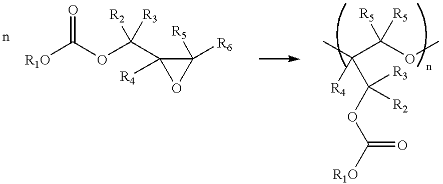

When polymerized, the carbonate may react readily with amines or diamines, to form graft and ladder copolymers containing an equal number of linear carbonate linkages for every alkanolamine linkage. The resins so formed behave as surfactants, and are useful in preparing agricultural formulations, personal care products, and coatings.

The following examples are illustrative of the present invention, and shall not be construed as limiting the scope of the invention in any way.

### EXAMPLE 1

3701.2 grams (40 moles) of ephichlorohydrin were charged in an autoclave with 30.0 grams of tetraethylammonium bromide, pressurized with 2288 grams (52 moles) of carbon dioxide, and heated to a temperature of 165° C. for three hours. During this three hour period, the autoclave pressure dropped from 1550 psig to 300 psig as carbon dioxide was incorporated into the final product molecule. After three hours, the reactor was cooled, and the resulting product (4-chloromethyl-1,3-dioxolan-2-one) was determined to be 98.93% pure by GC analysis. The resulting product was further purified by wiped film evaporation at a pressure of about 0.5 torr and a temperature of about 132° C. to obtain a final material that was 99.18% pure.

### EXAMPLE 2

200 mL of tetrahydrofuran (THF) was charged into a three-necked round bottom flask equipped with a thermocouple probe, a nitrogen inlet/outlet, an overhead stirrer, and a graduated dropping funnel. Then, 67.9 grams (0.497 moles) of 4-chloromethyl-1,3-dioxolan-2-one (prepared according to Example 1) was placed into the dropping funnel, and the system was thoroughly purged with nitrogen, at room temperature. Then, 52.4 grams (0.545 moles) of sodium t-butoxide was added to the flask, and stirring begun. The color of the contents of the flask was light yellow. After the addition of all of the sodium t-butoxide, the system was again purged with nitrogen for about thirty minutes, and the flask was cooled using a dry ice/acetone bath. Once the temperature of the contents of the flask had stabilized (to about −70° C.), 4-chloromethyl-1,3-dioxolan-2-one was slowly added drip-wise into the flask. Upon the addition of the 4-chloromethyl-1,3-dioxolan-2-one, the solution began to turn orange, and the temperature increased slightly, but was never allowed to exceed −60° C. After the addition of the 4-chloromethyl-1,3-dioxolan-2-one was complete, the flask containing the reaction mixture was maintained in the dry ice/acetone bath under stirring until the temperature of the solution ceased to rise. The dry ice/acetone bath was then removed, and the solution was allowed to slowly warm to room temperature. Once the solution warmed up to room temperature, the solution had a dark red color, and a salt-like precipitate was observed. The mixture was then stirred for an additional thirty minutes at room temperature, under nitrogen. Then, a predominant amount of the THF solvent was rotary evaporated, and the resulting product was isolated by extraction into ether in the presence of water. The organic phase was washed with three aliquots of water, dried (MgSO₄), and filtered. Subsequent concentration of the organic phase by rotary evaporation resulted in a light yellow liquid product. Final purification was by distillation at a temperature of 50° C. to 60° C., and a pressure of 0.1 mmHg to 0.5 mmHg to give a yield of pure t-butyl glycidyl carbonate of 70.3 grams. (81.1% of theoretical)

### EXAMPLE 3

Into a three-necked round bottom flask equipped with a thermocouple probe, a nitrogen inlet/outlet, a mechanical stirrer, and an addition funnel was charged 175 mL of tetrahydrofuran (THF). Then, 53.1 grams (0.389 moles) of 4-chloromethyl-1,3-dioxolan-2-one (prepared according to Example 1) was placed into the dropping funnel, and the system was purged with nitrogen for thirty minutes, at room temperature. Then 29.3 grams (0.430 moles) of sodium ethoxide was added to the THF while the mixture was stirred. After the addition of the sodium ethoxide, the system was again purged with nitrogen for about thirty minutes, and the flask was cooled using a dry ice/acetone bath. Once the temperature of the flask had stabilized at about minus 70° C., 4-chloromethyl-1,3-dioxolan-2-one was added drip-wise to the flask over the course of about 60 minutes, during the course of which the temperature of the flask contents were not permitted to rise above 50 degrees centigrade. Once the addition of the 4-chloromethyl-1,3-dioxolan-2-one was complete, the reaction mixture was slowly warmed to −20° Centigrade. After 30 minutes of stirring, the dry ice/acetone bath was removed, and the solution was allowed to gradually warm. After about ten minutes, the temperature of the mixture reached −5° C., and an ice bath was used to reduce the reaction exotherms. Even with the ice bath, the flask temperature quickly reached a maximum temperature of 30C after an additional ten minutes. After the temperature of the solution had cooled to room temperature, the solution was stirred for an additional thirty minutes under nitrogen. The THF solvent was then rotary evaporated and the resulting product was isolated using ether extraction in the presence of water. The organic phase was washed with three aliquots of water, dried (MgSO₄), and filtered. Further purification was achieved by distillation at a temperature of 50° C. to 60° C., and at a pressure of 0.1 mmHg to 0.5 mmHg to give a 23.4% yield of pure ethyl glycidyl carbonate.

In the reaction:

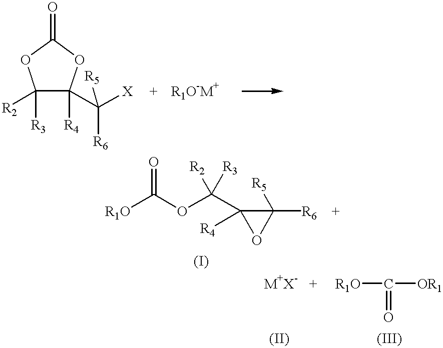

materials other than alkoxides are also useful as reactants R₁O⁻M⁺ in providing the alkyl glycidyl carbonate products of the invention, including without limitation phenolates, including those derived from alkylphenols, such as nonylphenol. Production of metallic phenolates has been known for quite some time by those skilled in the art by reaction of phenol or a mono-, di-, or tri-alkylated phenol with a basic species such as sodium hydroxide or sodium metal in a suitable solvent, such as an ether. Such phenolates or their solutions may be employed as a reactant according to the invention in the stead of a metallic alkoxide as described above, to yield a phenyl group in the position occupied by R₁ in equation 1 above. For those cases in which the phenolate chosen is a monoalkylated phenol whose alkyl group comprises any number of carbon atoms between about 8 and 25, the resulting alkyl glycidyl carbonates are good surfactants, suitable for use in various home and industrial laundry compositions, dishwashing compositions, personal care products, etc. (collectively “detergent composition”) or generally any other application where linear alkylbenzene sulfonate detergents are useful, in combination with other materials normally used in detergent composition formulations.

Solvents in which a process according to the present invention may be carried out include those having a sufficiently low freezing point so as to be liquid in the temperature range of about 0 degrees C. to minus 100 degrees C. However, solvents to be used in the invention should not have such a high boiling point that they are burdensome to remove by distillation upon purification of the product. Preferably the temperature range of the boiling point of the solvent is between 150° C. and 40° C. Preferred solvents are polar, aprotic solvents, and are well-classified as including those which are employable in the well-known process of solvent-phase anionic polymerization. Such a class includes, without limitation such solvents as: tetrahydrofuran, dimethylformamide, glycol ethers, diethyl ether, methyl tertiarybutyl ether, and the like. In cases where phenolates or alkylated phenolates are used as reactants R₁O⁻M⁺ in a reaction according to the invention, the preferred solvent is dimethylformamide.

Although various illustrative embodiments have been shown and described, a wide range of modification, changes, and substitution is contemplated in the foregoing disclosure, and one of ordinary skill in the art will appreciate the broad scope of the invention after reading this specification and the appended claims. In some instances, some features of the disclosed embodiments may be employed without a corresponding use of the other features. Accordingly, it is appropriate that the appended claims be construed broadly and in a manner consistent with the scope of the invention.

What is claimed is:

1) An alkyl glycidyl carbonate having the general structure: 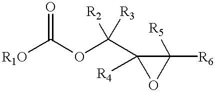
in which R₁ is selected from the group consisting of: any alkyl group having between 1 and 12 carbon atoms (inclusive), straight-chain or branched; or any aryl or alkyl aryl group having between 6 and 30 carbon atoms; and wherein R₂, R₃, R₄, R₅, and R₆ are each independently selected from the group consisting of: hydrogen; or any alkyl group having between 1 and 4 carbon atoms (inclusive), straight-chain or branched.

2) An alkyl glycidyl carbonate according to claim 1 in which R₁ is selected from the group consisting of: methyl, ethyl, propyl, isopropyl, n-butyl, t-butyl, phenyl, or alkylphenyl.

3) An alkyl glycidyl carbonate according to claim 2 wherein R₁ is a mono-alkylated phenyl group, wherein the alkyl group has any number of carbon atoms between about 8 and 25.

4) An alkyl glycidyl carbonate according to claim 2 wherein R₁ is a nonylphenyl group.

5) An alkyl glycidyl carbonate according to claim 1 in which R₂, R₃, R₄, R₅, and R₆ are all hydrogen.

6) An alkyl glycidyl carbonate according to claim 2 in which R₂, R₃, R₄, R₅, and R₆ are all hydrogen.

7) An alkyl glycidyl carbonate according to claim 4 in which R₂, R₃, R₄, R₅, and R₆ are all hydrogen.

8) An alkyl glycidyl carbonate according to claim 5 in which R₁ is selected from the group consisting of: methyl, ethyl, or tertiary butyl.

9) Tertiarybutyl, (2,3)-epoxypropyl carbonate.

10) Methyl, (2,3)-epoxypropyl carbonate.

11) Ethyl, (2,3)-epoxypropyl carbonate.

12) Phenyl, (2,3)-epoxypropyl carbonate.

13) Para-nonylphenyl, (2,3)-epoxypropyl carbonate.

14) A method of preparing an alkyl glycidyl carbonate comprising:
a) providing a reactant selected from the group consisting of: a metallic alkoxide or a metallic phenolate; and
b) contacting said reactant with a cyclic organic carbonate described by the structure: 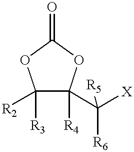
in which R₂, R₃, R₄, R₅, and R₆ are each independently a hydrogen or an alkyl group; and X is fluorine, chlorine, bromine, iodine, or any other leaving group within the context of nucleophillic substitution, in the presence of a polar, aprotic solvent.

15) A process according to claim 14 in which said solvent is selected from the group consisting of: ethers, glycols, amides, furans, or alcohols.

16) A process according to claim 14 in which said solvent is selected from the group consisting of: tetrahydrofuran, dimethylformamide, glycol ethers, diethyl ether, or methyl tertiarybutyl ether.

17) A process according to claim 14 in which said reactant is a metal alkoxide that is selected from the group consisting of sodium t-butoxide, potassium t-butoxide, lithium t-butoxide, sodium ethoxide, potassium ethoxide, lithium ethoxide, sodium methoxide, potassium methoxide, or lithium methoxide.

18) The method of claim 17, wherein the metal alkoxide comprises sodium t-butoxide.

19) The method of claim 14, wherein the cyclic organic carbonate comprises 4-chloromethyl-1,3-dioxolan-2-one.

20) A process according to claim 14 in which said other leaving group is selected from the group consisting of: phenoxy, alkylester, sulfate, sulfite, acetate, benzoate, or tosylate group.

21) A process according to claim 14, wherein the temperature of the solvent is less than about 20° Centigrade.

22) A process according to claim 14, wherein the temperature of the solvent is less than about 0° Centigrade.

23) A process according to claim 14, wherein the temperature of the solvent is less than about minus 20 degrees Centigrade.

24) The method of claim 14, wherein the suitable leaving group is selected from the group consisting of a phenoxy, alkylester, acetate, benzoate, or tosylate.

25) A process according to claim 14, wherein the reactant is present in a molar excess of between about 1.00% and 10.00% based upon the moles of cyclic organic carbonate present.

26) A process according to claim 14, wherein the cyclic organic carbonate comprises 4-chloromethyl-1,3-dioxolan-2-one, the reactant comprises an alkoxide salt, the solvent is selected from the group consisting of: tetrahydrofuran, methyl tert-butyl ether, diethyl ether, or ethylene glycol dimethyl ether, and the temperature of the solvent is maintained below about 20 degrees centigrade.

27) A process according to claim 14, wherein the cyclic organic carbonate comprises 4-chloromethyl-1,3-dioxolan-2-one, the reactant comprises a phenolate salt, the solvent is selected from the group consisting of: tetrahydrofuran, methyl tert-butyl ether, diethyl ether, or ethylene glycol dimethyl ether, and the temperature of the solvent is maintained below about 20 degrees centigrade.

28) A process according to claim 27 wherein said phenolate is derived from a monoalkylated phenol in which the alkyl group comprises any number of carbon atoms between 8 and 25.
